(19) United States
(12) Patent Application Publication
(10) Pub. No.: US 2013/0040296 A1
(43) Pub. Date: Feb. 14, 2013

(54) METHOD AND RAPID TEST DEVICE FOR DETECTION OF TARGET MOLECULE

(71) Applicants: Indrek Tulp, Tartu (EE); Janne Pullat, Valga (EE); Imre Mäger, Tartu (EE); Marko Lehes, Rae vald, Harjumaa (EE); Ülo Langel, Stockholm (SE)

(72) Inventors: Indrek Tulp, Tartu (EE); Janne Pullat, Valga (EE); Imre Mäger, Tartu (EE); Marko Lehes, Rae vald, Harjumaa (EE); Ülo Langel, Stockholm (SE)

(73) Assignee: SELFDIAGNOSTICS OÜ, Rae vald, Harjumaa (EE)

(21) Appl. No.: 13/575,771

(22) PCT Filed: Jan. 28, 2011
(86) PCT No.: PCT/IB11/00277

§ 371 Date: Oct. 22, 2012

(30) Foreign Application Priority Data
Jan. 29, 2010 (EE) P201000013

(51) Int. Cl. C12Q 1/68; C12M 1/34; G01N 21/64

(52) U.S. Cl. 435/6.11; 435/287.2

(57) ABSTRACT

Method and rapid test device for detection of target molecule from sample with which is performed purification and preprocessing of sample, preparation and modification, amplification, detection and capturing of target molecule, producing, of signal, amplification and visualization of read 5 out signal. For purpose of invention is used a device which includes in a case 1 one or many holes 2 for injection of sample, readout 4, target molecule detection nodal points attached to case 1 which includes one or many analysis chambers 3. Analysis chamber 3 is made of at least one room space 6, dividing wall 7, chamber 8 connected with 10 room space 6 with dividing wall 9 and at least one lever 5.

### FIELD OF TECHNOLOGY

The present invention belongs to the field of analysis and diagnostics. Precisely has been proposed a method for detection of target molecules and a rapid test device.

### BACKGROUND ART

Several patents address different rapid test devices and methods. Easiest and well-known are several rapid tests that determine pregnancy, narcotics etc. Well-known approaches employ for the sample analysis test-strips comprising different layers or test-sheets which appearance change color in case of identification specific antibody or antigene. This kind of application is utilized in different pregnancy tests. So far the most known applications base on lateral flow technology in multilevel strip format that appearance change color when reacts with a probe.

Different approaches used for explanation of different known applications.

European patent EP2031376 discloses a method for analysis more than one probe in liquid sample. The patent addresses a device involving separate zones and the described device has different zones that include several injection ports as well as several channels for detection one ore more analytes from liquid probe. The signal is detected electronically.

U.S. Pat. No. 6,203,757 discloses a method for simultaneous detection of multiple analytes. At one side of test device is located an entry port for injection of a sample. The sample is delivered then into zones within a test device. Every zone is equipped with a screen for displaying a detection read. The method basis on immunochromatographic strips (i.e. rapid tests for pregnancy etc.) and do not include distinct chambers for sample processing. European patent EP1595953 discloses a method of a signal amplifaction where a mutated gene is amplified.

US patent US2005214796 discloses enhancing probe quantity and signal detection through exploitation of dendrimeres. This patent addresses a synthesis of oligonucleotide probes and mass spectrometry to target homologous segments.

Here is presented a solution for the production of oligonucleotide copies as well as an example of detection signal promotion and amplification. However the detection is performed by mass-spectrometry and thus is not meant for use in rapid tests.

Conventional methods for the DNA analysis from urine are well known too. For example covers international patent WO2008021995 a method that basis on PCR technology that is used for obtaining a nucleic acid, purification and analysis from a biological sample. From urine viral nucleic acids (DNA and RNA) are detected. Mainly has been focused on preprocessing and purification of a sample. The method is not meant for the rapid tests application as it exploits PCR method for the amplification.

So far is the biggest deficiency of known methods is their limited use at home or without medical education for the analysis by single sample insertion more than one diseases. Thus, the known solutions have limited options for use as devices and a methods and could be used only for given purposes to detect either disease or existence or absence of a certain substance.

The main imperfection of rapid tests that base on antibody detection is their objectivity. For the detection certain antibodies has patient got ill and corresponding immune reaction activated. Hereby is not possible to detect disease in early stage, when antibodies absent and test provides false-negative result. Also the situation when patient has convalesced but organism still contains antibodies at higher level that the false-positive result can be detected. Detection platforms basis on nucleic acids are currently in use only at laboratories as they need specific and expensive equipment.

### BACKGROUND OF THE INVENTION

The aim of present invention is a new method and device for the detection target molecule from sample in rapid tests. Additional purpose of present invention is to propose a compact solution that excludes previously mentioned deficiencies, including lower production cost as well as easy-to-use principle.

Method according to the present invention comprises a combination of several steps including preprocessing a sample and target molecule for analysis, amplification and detection of target molecule, production and amplification of signal.

Current method can be applied in medical diagnostics for detection of diseases, narcotics or indefinite target molecule. The method can be used for detection of target molecule from different liquids, including human body liquids (i.e. urine, saliva, blood, sperm, secretions of nose etc.) and other biological liquids.

The present invention presents a device for application of a described method(-s). The aim is to provide a device for a rapid test that is suitable to use for people without medical education and in the situation where is necessary to analyze samples in big amounts.

Rapid test device in present invention contains at least one entry port for the probe insertion, one or more chambers for sample analysis, one or more movable levers, one or more detection reads for the presentation of result. Analysis chamber for detection is divided in order to process steps into one or several rooms that are separated with membranes. Every room is united with an additional chamber for solvents and reagents. Mechanically movable lever inside the analysis chamber is meant for the breaking of intermediate wall between one or more rooms and corresponding additional chamber for leading solvent or reagent into the reaction room

### LIST OF THE DRAWINGS

Present invention is explained by referred drawings, where

FIG. 1 presents schematically visualization of a rapid test device in accordance to present invention;

FIG. 2 presents on FIG. 1 visualized a scheme of rapid test analysis chamber;

FIG. 3 presents on FIG. 1 visualized analysis chamber scheme of rapid test according to the one alternative implementation example.

### EXAMPLE OF IMPLEMENTATION

In accordance of the present invention, a rapid diagnosis device is designed for the multiplex and/or simultaneous detection and/or analysis one or more target-molecules and/or marker(s) in a sample comprises housing 1, one or two entry ports for the probe insertion 2, into the device housing 1 is located one or more chambers for the analysis 3, one or more readers 4, one or more levers 5.

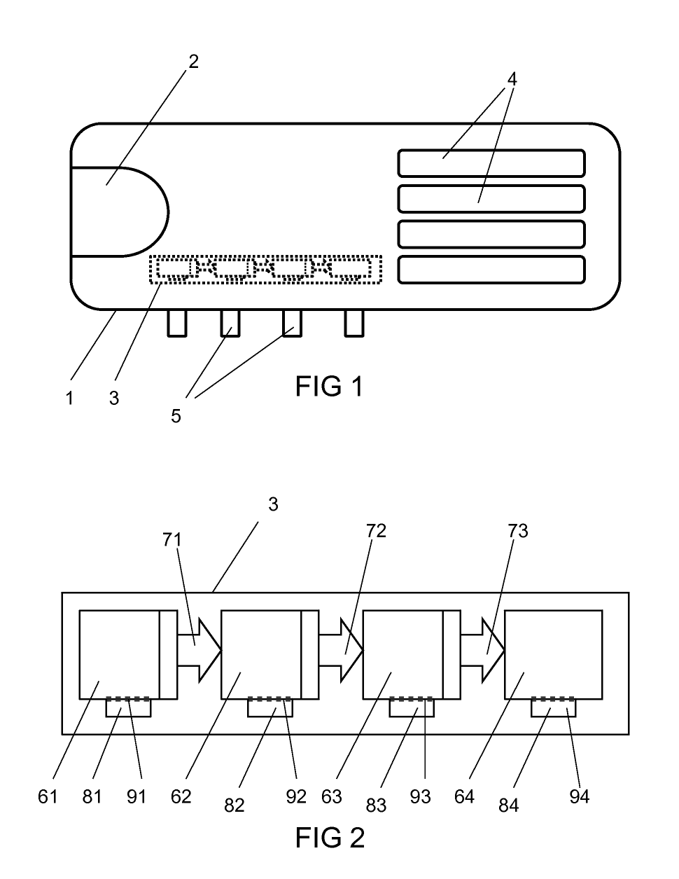

FIG. 1 shows a plan view of a device housing of a rapid diagnosis device in accordance with an embodiment of the present invention; demonstrated is one opening for the insertion of a probe, four readings of results 4 and two levers 5.

Analysis chamber 3 is designed in several parts having different functions and/or for at least one or more different consecutive or several consequently positioned rooms that are united with each other by at least one nanotube or at least one nanocapillary or at least membrane or intermediated by their combination.

FIG. 2 shows analysis chamber 3 that comprises a room 61 for sample preprocessing according to the method in present invention, first amplification room 62, capture molecules matrix containing room 63 and second amplification room 64. Rooms 61, 62, 63 and 64 are integrated with each other through intermediates 71, 72 and 73. At each room is located additional chambers 81, 82, 83, 84 for solvents and reagents, whereby intermediate walls 91, 92, 93, 94 of rooms 61, 62, 63, 64 and corresponding additional chambers 81, 82, 83, 84 are prepared from a material that can be easily shattered. For the breakage of intermediate walls 91, 92, 93, 94 is at least one lever 5 necessary, whereas the breaking of the wall could be performed mechanically, electrically, chemically etc.

Within a preprocessing chamber 61 is performed at least one of the following steps:
preprocessing of sample insertion;
preprocessing of buffered sample solution;
preprocessing of capture molecule in the sample.

Within amplification chamber 62 is performed at least one of the following steps:
amplification of a target molecule in the sample;
amplification of a marker(-s).

Within chamber 63 that include a functional matrix of capture molecules is performed at least one of the following steps:
capture or binding of a specific target molecule(-s);
capture or binding of a marker(-s).

Within a second amplification chamber 64 is performed an amplification of a detection signal.

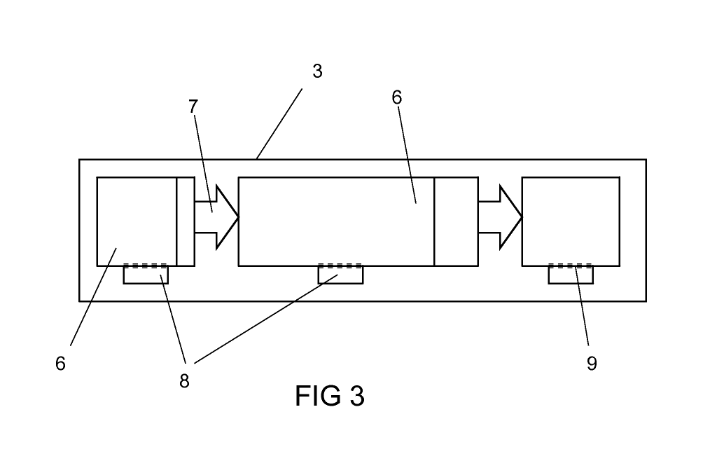

FIG. 3 shows alternative rapid test analysis chamber 3 where the processes of chambers 62 and 63 are combined.

Though is clear for the specialists of this field that the rapid test device in present invention comprising a number of entry ports 2, analysis chamber 3, its' components 6, detection read 4, lever 5, intermediate 7, additional chamber 8 and dividing wall 9 and their combinations are not limited to the examples listed above.

The molecule(-s) in the functional array matrix that binds target molecule(-s) and/or marker(-s) specifically can be DNA and/or RNA and/or PNA and/or L-DNA and/or LNA and/or antibody and/or protein. The detection of target molecule(-s) and/or marker(-s) from the biological sample in rapid test may be performed through amplification of a signal by nanoparticles and/or dendrimere(-s) and/or dendrimeric structures and/or complementary DNA and/or PNA and/or L-DNA molecule(-s) at the defined position of a molecule matrix.

Present invention involves detection of target molecule from sample by DNA spiral structure(-s) sequence specifically hair-pin structure (element of secondary DNA structure) forming polyamides.

Current method involves additionally in situ amplification of a target molecule from a sample. In order to detect and/or analyze DNA and/or RNA from sample in functional rapid tests a target molecule is amplified by double-stranded DNA spiral structure(-s) with an assistance of oligonucleotide(-s) according to triple helix formation. The specific target probe is enzymatically preprocessed and/or ligated before its capture by a functional matrix molecule. Preprocessed target molecule and/or marker bind specifically to the matrix molecule and a double-stranded complex is formed. Obtained double-stranded molecule complex anneals to complementary biotin-labeled PNA molecule, forming thus depending on sequence either PNA₂/DNA and/or PNA/DNA₂ and/or PNA₂/DNA₂ complexes. The biotin attached to the PNA molecule is visualized and/or amplified as a detection signal by nanoparticles and/or dendrimeres and/or dendrimeric structures.

The present invention involves binding a target molecule and/or marker to the anti-biotin antibody conjugated nanoparticles and thereafter attachment of DNA and/or RNA in sample to the biotin and/or marker(-s) for detection and/or analysis by single molecular label.

The rapid test diagnosis method of current invention includes additionally a conjugation of a target molecule in a sample with specific molecule and its' further elution via nanotube(-s) and/or capillary(-es) and/or membrane(-s) to the test strip where the results get visualized through color.

The rapid test diagnosis method of current invention includes additionally in situ amplification of a target molecule in a sample and/or signal basis on Autonomic DNA and/or RNA nanomachine technology. In that case a short DNA probe anneals to a target DNA and/or RNA. The probe then acts as a primer for a Rolling Circle Amplification reaction. The free end of the probe anneals to a small circular DNA template. A DNA or RNA polymerase is added to extend the primer. The DNA or RNA polymerase extends the primer continuously around the circular DNA or RNA template generating a long DNA or RNA product that consists of many repeated copies of the circle. By the end of the reaction, the polymerase generates many thousands of copies of the circular template, with the chain of copies tethered to the original target DNA or RNA. This allows for spatial resolution of target and rapid amplification of the signal.

The present invention involves also in situ amplification of a detection signal that is necessary for rapid test analysis of target molecule in sample basis on a autocatalytic DNA nanomachine technology. Autocatalytic DNA and/or RNA nanomachine is promoted by a formation of a target sequence molecular link. DNA and/or RNA polymerase bounds on this complex and synthesizes a complementary target sequence and/or copy sequence that is marker-specific. Copy of target molecule and/or marker that is synthesized and released in an autocatalytic DNA and/or RNA nanomachine promotes a new repetition of synthesis process and thus itself amplifies a detection signal.

For the additional explanation of present invention are examples presented that describe a detection of target molecule from different body liquids.

### EXAMPLE 1

Detection of Chlamydia RNA. The sample solution is buffered for optimal reaction environment. RNA strands are fragmentized by restrictase enzymes. These RNA molecules will be amplified basis on continuous isothermal enzymatic rolling amplification (RCA—Rolling Circle Amplification) method. For the amplification of detectable signal the nanoparticles (molecular beacon) are used. Preprocessed fragment is amplified basis LAMP method by DNA polymerase BST and assembles with four specially designed oligoprimers at temperature 60° C. to 65° C.

In order to maintain pH at optimal level 7.6 to 8.0 a Tris-solution is added to the urine solution and restrictase enzymes BamH I and Sal I that cut mRNA specifically into shorter fragments. Into preprocessed solution are added reagents like Chlamydia mRNA specific primers, nucleotides and reverse transcriptase. Reaction is isothermal (at temperature range 35 to 38° C.). In the first step of the amplification, a promoter-primer hybridizes to the mRNA at a defined site, reverse transcriptase creates a copy DNA and a RNA:DNA duplex is formed. Primer 2 anneals to the copy DNA and reverse transcriptase synthesizes a new copy, the double-stranded DNA molecule is obtained in the end. DNA complex is denaturized and complementary DNA that contains molecular particles is added. This DNA probe hybridizes specifically to the copy DNA. In range of 150 to 250 μl particles buffered solution (1 mM MgCl2, 20 mM Tris-HCl, pH 8.0) excited at 491 nm and emission is at 515 nm.

### EXAMPLE 2

Detection of a Hepatitis virus DNA in blood. In order to maintain a preferable reaction environment a buffer is added to the solution. In the buffered is performed a fragmentation of DNA molecules. Cleaved hepatitis virus DNA sequence sites amplified by Transcription Mediated Amplification (TMA) mechanism and followed by DNA detection according to the autonomous DNA nanomachines technology. Detected signal is amplified by dendrimeric molecules.

In order to maintain pH in a range 7.6 up to 8.0 for the stabilization purposes of solution and restrictase enzyme Bsp 191, that cuts DNA specifically into specific sites, a phosphate buffer (H₂PO₄⁻: HPO₄²⁻) is used. Following reagents have inserted into preprocessed sample—Hepatitis DNA sequence specific promoter primer, nucleotides and DNA polymerase. Reaction is performed isothermally at 37° C. Promoter primer binds specifically to DNA sequence. Reverse transcriptase creates a DNA copy of the target sequence and double-stranded DNA fragments are produced. The specific target sequence that is synthesized and released in the autocatalytic DNA nanomachine promotes a repetition of synthesis cycle polypropylene amine dendrimere that is covered with 32 dansyle units.

### EXAMPLE 3

Detection of Herpes virus in saliva. To the human saliva containing solution is added sodium hydroxide (NaOH) or carboxyl acid consisting Tris(hydroksymethyl)aminomethane or carboxyl acid buffer in order to maintain pH at optimal level pH>6.9 and restrictase enzyme Bsp 191 that will cut DNA specifically into shorter fragments. Preprocessed fragment will be amplified basis on LAMP method by DNA polymerase Bst and interaction of specially designed four oligoprimers at temperature range of 60° C. to 65° C. These four primers are designed according to 6 different specific DNA sequences. Oligonucloetides that form triple helices are added for detection of amplified DNA fragments by TFO method. Mixture of FITC conjugated and unlabeled oligonucleotides (1:1) in Tris-EDTA buffer (TE buffer, 10 mM Tris (pH 7.4), 1 mM EDTA) is incubated at 63 to 67° C. for 15 minutes and then cooled at RT. A double-stranded DNA complex is incubated with 10 μl of 10 mM Tris (pH 7.4), 1 mM spermidine and 20 mM MgCl₂ for 2 hours at 37° C. For the detection of FITC molecules that are hybridized to the DNA duplexes the pH is adjusted to the 9.5 by addition of 500 mM carbonate buffer and detected at excitation of 490 nm and emission at 513 nm by flow cytometry.

### EXAMPLE 4

Detection of Gonorrhea in urine. Pathogenic bacteria are lysed (i.e. solution of 0.1% Triton-X100) and in order to maintain optimal reaction environment a buffer (Tris-solution, preferably at a range pH 7.6 to 8.0 for the solution stability) is added. Double-stranded DNA of bacteria will be cut by restrictases BamH I and Sal I into shorter fragments if necessary. In assistance of PNA clamps two regions of double-stranded DNA will be opened and labeled oligonucleotide primer can hybridize on a DNA strand that will capture the pathogenic sequence on a solid surface. On another opened DNA strand hybridizes a sequence through the linear DNA circle structure (i.e. RCA) and initiates amplification. From the pathogens consisting surface will be removed unnecessary components and RCA buffer and reagents will be added. Depending on primers either linear or exponential RCA is used. In result will be attached long single-stranded DNA or several linear DNA strands involving tree onto the surface.

Alternatively, depending on RCA promoter design and use of compatible endonuclease, could be obtained also single-stranded short DNA fragments in the solution. Products will be detected according to labeled DNA primers (i.e. gold or different label that enables exploitation of enzymatic amplification method for detection).

### EXAMPLE 5

Detection of Ureaplasma in semen. In order to maintain a semen containing solution at stabile pH level a Tris-solution is added, preferably pH range 7.6 to 8.0. The DNA will be ligated by restrictes BamH I and Sal I into sohrter fragments if necessary. In assistance of PNA clamps two regions of double-stranded DNA will be opened and labeled oligonucleotide primer can hybridize on a DNA strand that will capture the pathogenic sequence on a solid surface. On another opened DNA strand hybridizes a sequence through the linear DNA hairpin structure sequence (i.e. HCR (Hybridization Chain Reaction) and promotes reaction. From the pathogens consisting surface will be removed unnecessary components and HCR buffer and reagents will be added. Depending on primers either linear or exponential RCA is used. In result will be attached long single-stranded DNA or several linear DNA strands involving tree onto the surface. Alternatively, depending on RCA promoter design and use of compatible endonuclease could be obtained also single-stranded short DNA fragments in the solution. Products will be detected according to labeled DNA primers (i.e. gold or different label that enables exploitation of enzymatic amplification method for detection).

### EXAMPLE 6

Detection of Listeria monocytogenes in vaginal secretion. Vaginal secretion is collected on a little stick with a cotton wool tip and added to the buffered solution (preferably Tris-solution at a range of pH 7.6 to 8.0 to maintain solution stability). Cells will be lysed (i.e. with solution of 0.1% Triton-X100). Pathogenic double-stranded DNA is ligated by restrictases if necessary. Two reverse primers that hybridize with pathogenic DNA are added if recombinase is presented. For detection is one of primers labeled (i.e. gold or label necessary for the enzyme assisted amplification), another molecule is assembled with molecule that captures a reaction product onto the solid surface. Addition of DNA polymerase promotes isothermal reaction and big amount of copies of double-stranded with pathogenic DNA fragments. These fragments with certain length could be transferred by lateral flow methods to the test-strips and detect according to the conventional methods.

### EXAMPLE 7

Detection of Syphilis in blisters secretion. Blisters sample is collected on a little stick with a cotton wool tip and added to the buffered solution (preferably Tris-solution at a range of pH 7.6 to 8.0 to maintain solution stability). Pathogenic organisms will then captured by specific antibodies to the solid surface. These antibodies will then be labeled with secondary antibodies that are attached to sequence of linear DNA strand circle-structure (i.e. promoter of RCA reaction). From the pathogenic DNA consisting surface will be removed unnecessary components and RCA buffer and reagents will be added. Depending on primers either linear or exponential RCA is used. In result will be attached long single-stranded DNA or several linear DNA strands involving tree onto the surface. Alternatively, depending on RCA promoter design and use of compatible endonuclease could be obtained also single-stranded short DNA fragments in the solution. Products will be detected according to labeled DNA primers (i.e. gold or different label that enables exploitation of enzymatic amplification method for detection).

### EXAMPLE 8

Detection of Trichomoniasis in penile secretion. Penile secretion is collected on a little stick with a cotton wool tip and added to the buffered solution (preferably Tris-solution at a range of pH 7.6 to 8.0 to maintain solution stability). Pathogenic bacteria will then captured by specific antibodies to the solid surface. These antibodies will then be labeled with secondary antibodies that are attached to DNA sequence of hairpin-structure (i.e. promoter of HCR reaction).

From the pathogens consisting surface will be removed unnecessary components and HCR buffer and reagents will be added. Depending on primers either linear or exponential RCA is used. In result will be attached long single-stranded DNA or several linear DNA strands involving tree onto the surface. Alternatively, depending on RCA promoter design and use of compatible endonuclease could be obtained also single-stranded short DNA fragments in the solution. Products will be detected according to labeled DNA primers (i.e. gold or different label that enables exploitation of enzymatic amplification method for detection).

1. A method for detecting target-molecule in a sample comprising the steps of:
(a) injection of a sample, purification and preprocessing, wherein lysing, stabilizing and other beneficial reagents and solutions are enclosed into sample;
(b) detection of a target-molecule, wherein target-molecule is enzymatically or chemically modified into suitable foul' in order to interact with a capture molecule and amplify when it is necessary;
(c) a read-out signal is produced by a capture molecule or compound that interacts with it, which is characterised by step
(b) where enzymatically ligated target molecule (nucleic acid—RNA, DNA) is amplified isothermally and obtained products are detected by complementary nucleic acid oligomere and within the step
(c) where detectable signal is produced by complementary nucleic acid oligomere that is chemically attached or with specifically interacting particle.

2. Method according to claim 1, which is characterised by that the step (b) where target molecule interacts with specific capture molecule and different specific molecule (DNA, RNA, PNA etc. oligomere) is released and amplified.

3. Method according to claim 1, which is characterised by that the step (b) where target molecule interacts with specifically different molecule that contains property for signal amplification like dendrimeres, FITC or gold-labeled etc. enzymatic amplification method.

4. Method according to claim 1, which is characterised by that the step (b) where one or more specific target sequences are detected.

5. Method according to claim 1, which is characterised by that the step (c) where detectable signal is produced by molecular particle is chemically bound or specifically interacting.

6. Method according to 1, which is characterised by that the step (c) where detectable signal is produced by chemically bound or specifically interacting FITC molecule.

7. Method according to claim 1, which is characterised by that the step (c) where detectable signal is produced by chemically bound or specifically interacting labeled enzymatic amplification method.

8. Rapid test device implemented for detection of target molecule in a sample contains housing (1), one or more entry ports (2), one or more detection reads (4) differs by location of detection junctions that are bound into housing (1), and comprises one or more analysis chambers (3) and integrates at least one room (6), intermediate (7), room (6), via dividing wall (9) united additional chamber (8) and at least one lever (5).

9. Rapid test device according to claim 8, which is characterised by that the rooms (6) are located consequently within analysis chamber (3).

10. Rapid test device according to claim, which is characterised by that the lever (5) is connected to the dividing wall (9).

11. Rapid test device according to claim 8, which is characterised by that dividing wall (7) is integrated from a one nanotube or at least from one nanocapillary or at least from one membrane.

12. Rapid test device according to claim 8, which is characterised by that the entry port (2) is absorbent.

13. Rapid test device according to claim 9, which is characterised by that the lever (5) is connected to the dividing wall (9).
